(19) United States
(12) Patent Application Publication
(10) Pub. No.: US 2005/0098776 A1
(43) Pub. Date: May 12, 2005

(54) Preparation of fullerenol having nanolayer or nanowire structure

(76) Inventors: K. E. Geckeler, Gwangju (KR); Yulan Wang, Gwangju (KR)

Correspondence Address: ANDERSON, KILL & OLICK, P.C., 1251 AVENUE OF THE AMERICAS, NEW YORK,, NY, 10020-1182, US

(21) Appl. No.: 10/965,581

(22) Filed: Oct. 14, 2004

(30) Foreign Application Priority Data
Oct. 14, 2003 (KR) 2003-0071563

(51) Int. Cl. H01L 29/08; H01L 35/24; C07C 39/12

(52) U.S. Cl. 257/40; 568/732

(57) ABSTRACT

Fullerenols having a nanolayer or a nanowire structure are prepared under a mild condition with high efficiency by reacting fullerene with an alkali metal hydroxide dissolved in water.

### FIELD OF THE INVENTION

The present invention relates to a method for preparing a fullerenol having a nanolayer or nanowire structure.

### BACKGROUND OF THE INVENTION

Multi-hydroxylated fullerenes, fullerenols, have unique structural and physiochemical properties and they have been used in various applications such as scavengers of oxygen, hydroxyl or superoxide radicals; a proton conductor in fuel cells; a spherical molecular core in the design of dendritic and star-shaped polymers; and a building block in fabricating conducting elastomers. It has been reported C₆₀-KOH adducts prepared via adding hydroxyl to C₆₀ fullerene in toluene, but the adducts are unstable in the presence of oxygen (see Naim, A. et al, Tetra. Lett. 1992, 33(47), 7097-7102). Li et al. reported the addition of hydroxyls to C₆₀ molecule in benzene in the presence of a phase transfer catalysts (tetrabutylammonium hydroxide) under air (see Li, J. et al. J. Chem. Soc., Chem. Commun. 1993, 23, 1784-1785).

Lately, the preparation of fullerenols was carried out not by the direct addition of hydroxyl to C₆₀ fullerene, but by other indirect processes, which involve forming substituted derivatives of fullerene in an acid medium, followed by replacing the substituents with hydroxyl groups to form fullerenols.

However, conventional methods of preparing fullerenols are complicated, uneconomical, and environmentally unfriendly.

### SUMMARY OF THE INVENTION

Accordingly, it is a primary object of the present invention to provide a simple and efficient process for preparing fullerenols having improved physical properties.

In accordance with the present invention, fullerenols are prepared by reacting fullerene with an alkali metal hydroxide dissolved in water.

### BRIEF DESCRIPTION OF THE DRAWINGS

The above and other objects and features of the present invention will become apparent from the following description of the invention, when taken in conjunction with the accompanying drawings, which respectively show:

FIGS. 1A to 1B: Infrared (IR) spectrum of C₆₀ (a), fullerenols prepared in Example 1 (b), acetyl fullerenols (c), and acetyl fullerenol-2,4-dinitrophenyl-hydrazone (d);

FIG. 1C: Solid-mass spectrum of fullerenols;

FIG. 2: X-ray photoelectron spectra (XPS) of fullerenols (A); C 1s region curve fitting (B); and O 1s region curve fitting (C) (The dentate curves are the originals and the smooth curves are the results of curve fitting);

FIG. 3: Differential thermal analysis and thermogravimetric analysis (DTA-TGA) curve of fullerenols prepared in Example 1 and C₆₀; and

FIG. 4: Scanning electron microscopy (SEM) image of fullerenols prepared in Example 1.

### DETAILED DESCRIPTION OF THE INVENTION

In accordance with the present invention, fullerenols having improved physical properties may be prepared under gentle conditions in a high yield, by reacting fullerene with an alkali metal hydroxide dissolved in water.

In a preferred embodiment of the present invention, the reaction is carried out at a temperature in the range of 50 to 150° C.

In a preferred embodiment of the present invention, about 0.3 to 1 mol of alkali metal hydroxides dissolved in 5 to 100 ml of water are reacted with about 1 mmol of fullerene.

In a preferred embodiment of the present invention, the alkali metal hydroxide may be KOH or NaOH.

In another preferred embodiment of the present invention, the process may further comprise the step of adding an additional amount of water during the reaction.

In a preferred embodiment of the present invention, the process may further comprise a step of separating a solid product from the reactant, and washing and drying the product. Preferably, the product is dried at a temperature in the range of 50 to 150° C.

The present inventor thought of exploring OH⁻ as a nucleophilic agent which directly react with C═C groups of fullerene in water. The reaction mixture is heated until no black solid of fullerene is visible at the bottom of the reactor when the reaction mixture becomes brown-colored. The residual alkali metal hydroxides is washed off with water, and then dried overnight, the results show fullerenols with nanolayer and nanowire structures and remarkable properties in good yields and high purity. The supernatant basic reaction solution can be used for the next reaction solvent, thus a certain kind of recycling is possible; that is another advantage of the technology present over previous technologies.

The present invention is further described and illustrated in the following Examples, which are, however, not intended to limit the scope of the present invention.

### EXAMPLE 1

2.3503 g of KOH was dissolved in 1.5 ml of H₂O in a 10 ml glass reactor while stirring at room temperature, and 58.2 mg (0.08 mmol) of C₆₀ was added thereto. The mixture was heated to about 120° C. until no black solid of C₆₀ was visible at the bottom of the reactor when the reaction mixture became brown-colored (about 13 hours). Then, 2 ml of water was added thereto while continuing the reaction for another 3 hours. The reaction mixture was allowed to cool to room temperature and centrifuged, to obtain a dark-brown solid, which was washed with water until the supernatant solution became neutral. The dark-brown solid was dried at 70° C. overnight, to obtain 825.6 mg of a product (98% yield). Empirical formula: C₆₀H₂₉O₁₉, purity 96.3% based on the XPS results (C 1s 75.12%, O 1s 21.19%).

### EXAMPLE 2

3.0 g of KOH was dissolved in 2.0 ml of water in a 10 ml glass reactor while stirring at room temperature, and 60 mg (0.083 mmol) of C₆₀ was added thereto. The mixture was heated to about 120° C. until no black solid of C₆₀ was visible at the bottom of the reactor when the reaction mixture became brown-colored (about 13 hours). Then, 2 ml of water was added thereto while continuing the reaction for another 3 hours. The reaction mixture was allowed to cool to room temperature and centrifuged, to obtain a dark-brown solid, which was washed with water until the supernatant solution became neutral. The dark-brown solid was dried at 70° C. overnight, to obtain 856.5 mg of a product (98% yield).

The structure, chemical functional groups, composition and properties of the fullerenols prepared in Examples 1 and 2 were determined based on the results of scanning electron microscopy (SEM) images, X-ray photoelectron spectra (XPS), infrared (IR) spectra, mass spectrum (MS), derivative studies, differential thermal analysis and thermogravimetric analysis (DTA-TGA).

(1) IR Spectra

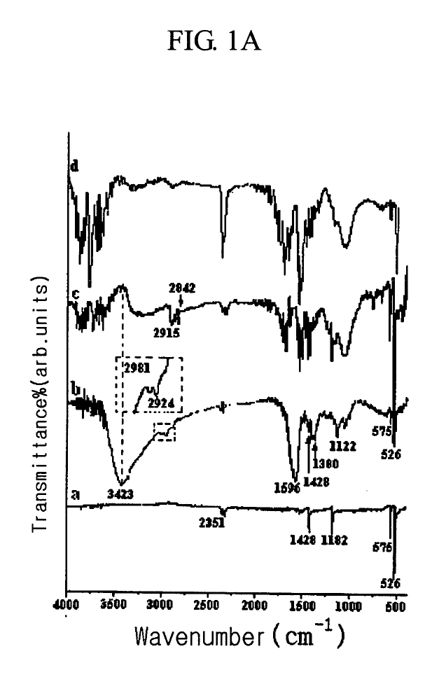

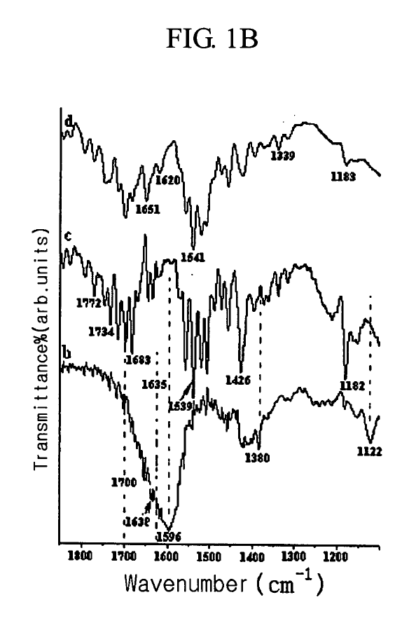

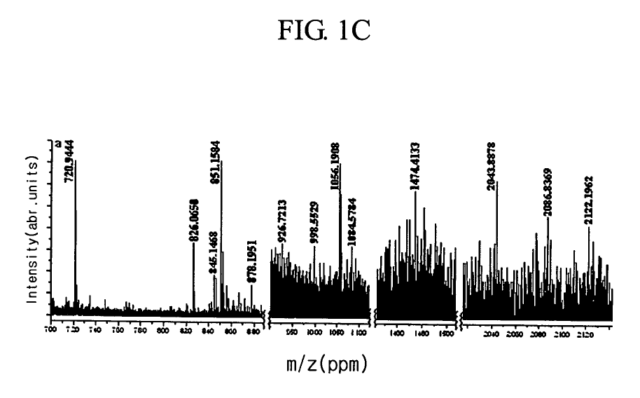

The IR spectra of C₆₀, fullerenols, acetyl fullerenols and acetyl fullerenol-2,4-dinitrophenylhydrazone are shown in a, b, c and d of FIG. 1A, respectively. For comparison, peaks of respective hydrazones at 1850-1100 cm⁻¹ are also shown in b, c and d in FIG. 1B. Comparing the IR scan of fullerenols (FIG. 1A, b) with that of the starting material, C₆₀ (FIG. 1A, a), fullerenols show new peaks at 3423 cm⁻¹, 2981 cm⁻, 2924 cm⁻, 1638 cm⁻¹, 1380 cm⁻¹, 1122 cm⁻¹ and about 1596 cm⁻¹. The characteristic peaks at 1428 cm⁻¹, 1182 cm⁻, 575 cm⁻ and 526 cm⁻¹ of C₆₀ are all intact in the fullerenols scan, which means that the basic framework of C₆₀ is retained in the fullerenols. The new peaks at 3423 cm⁻, 2981 cm⁻, 2924 cm⁻, 1596 cm⁻, 1380 cm⁻, 1122 cm⁻¹ are considered to be due to the hydroxyl groups (—OH) of the fullerenols. In general, α, β, α′, β′-unsaturated and diaryl ketones show an absorption peak in the rage of 1644-1623 cm⁻¹. The new peak at 1638 cm⁻¹ is attributed to the existence of ketone groups (—C═O or —O—C—O—) in fullerenols.

In order to confirm the presence of hydroxyl groups in fullerenols, acetyl fullerenols were synthesized by acetylation of fullerenols with acetic anhydride. Comparing the IR spectrum of fullerenols (FIGS. 1A and 1B, b) with that of acetyl fullerenols (FIGS. 1A and 1B, c), three differences are clearly seen: first, the broad and strong peak at 3423 cm⁻¹, the strong peak at 1596 cm⁻¹, the moderate peak at 1380 cm⁻¹ present in the IR spectra of fullerenols, which are attributable to —OH groups, all disappear in the IR spectra of acetyl fullerenols; second, the peaks above 1700 cm⁻¹ are intensified in the IR scan of acetyl fullerenols, especially the peaks at 1734 cm⁻¹ and 1772 cm⁻¹; and third, new peaks at 1182 cm⁻¹ (C—O in ester), 2915 cm⁻¹ (C—H stretching frequency), 2842 cm⁻¹ (C—H stretching frequency) appear for —COCH₃ groups are observed for acetyl fullerenols. Accordingly, the fact that fullerenols indeed contained hydroxyl groups is confirmed.

In addition, it is also clearly seen the peaks at 1635-1700 cm⁻¹ for ketone groups (—C═O or —O—C—O—) in fullerenols are retained in acetyl fullerenols. In order to confirm the presence of ketone groups in fullerenols, acetyl fullerenol-2,4-dinitrophenyl-hydrazone was synthesized by the reaction of acetyl fullerenol with 2,4-dinitrophenylhydrazine in aqueous HCl. In the IR spectrum of this derivative (FIG. 1B, d), the peaks at 1651 cm⁻¹ (C═N stretching), 1620 cm⁻¹ (C—N stretching), 1541 cm⁻¹ (NO₂ asymmetric stretch) and 1339 cm⁻¹(NO₂ asymmetric stretch) are evident, strongly supporting the existence of ketone groups in fullerenols.

(2) Mass Spectrum (MS)

The basic peak at δ720.9444 in MALDI-TOF (matrix-assisted laser desorption/ionization-time of flight) solid-mass spectrum (solid-MS, FIG. 1C) of fullerenols is in accord with the framework of fullerenols, and other peaks at δ826.0650, 845.1468, 851.1584, 878.1951, 926.7213, 998.5529, 1056.1908, 1474.4133, 2043.8874, 2087.8369 and 2122.1962 are assigned to C₆₀H₁₀O₆⁺, C₆₀H₁₀O₄(OH)₃⁺, C₆₀H₃O₈⁺, C₆₀H₁₀O₅(OH)₄⁺, C₆₀H₁₀O₈(OH)₄⁺, C₆₀H₁₀O₅(OH)₁₀⁺ .H₂O, C₆₀H₁₉O₉(OH)₁₀⁺ .2H., HOC₆₀-C₆₀C₆₀OH⁺, C₆₀H₁₈O₉(OH)₁₀-C₆₀H₉O₉(OH)₇⁺, C₆₀H₁₈O₉(OH)₁₀-C₆₀H₁₈O₉(OH)₉⁺.2H. and C₆₀H₁₈O₉(OH)₁₀-C₆₀H₁₈O₉(OH)₁₀⁺ .H₂O, respectively.

(3) X-Ray Photoelectron Spectra (XPS)

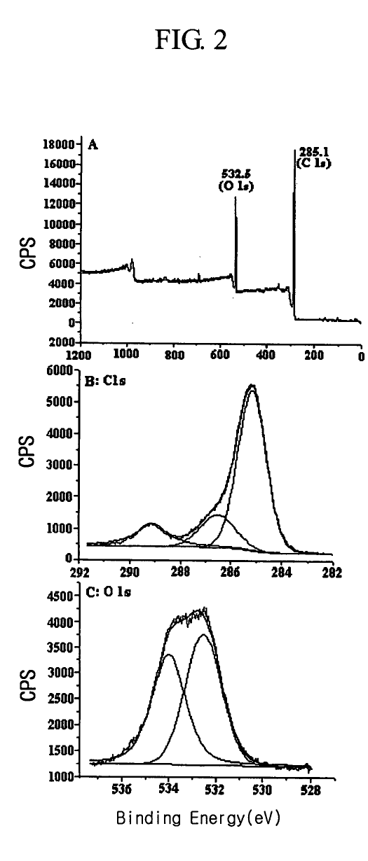

The X-ray photoelectron spectra (XPS) studies were conducted to assess the elemental composition of fullerenols (FIG. 2). The XPS data obtained for the oxy C₆₀ nanospheres are shown in Table 1.

TABLE 1
XPS analysis for fullerenols

| | Central peak | FWHM | Percent | Element No. |
|---|---|---|---|---|
| Peak | BE (eV) | (eV) | (%) | In Mononer |
| O 1s | 532.50 | 2.903 | 21.19 | 19 |
| C 1s | 285.10 | 2.170 | 75.12 | 60 |
| F 1s | 689.70 | 1.915 | 2.67 | — |
| Si 2p | 104.55 | — | 1.02 | — |

The number of carbon atoms of fullerenols is set at 60 based on the assumption that the C₆₀ framework is unchanged in fullerenols, and the number of oxygen atoms of fullerenols is calculated to be 19 based on the data of XPS analysis. The Si 2p signal (1.02%) may originate from the glass reactor which could have undergone some reaction with concentrated KOH at a high temperature, and F (2.67% ) can be traced to the teflon plastic centrifuge container in which isolation, washing, and drying steps were all carried after the reaction of C₆₀ with KOH. If the total percentage of C and O could represent the purity of fullerenols, the XPS results show that the purity of the fullerenols (96.28%) thus prepared is higher than that (93%, C 1s 58%, O 1s 35%) synthesized by a previous method.

Curve fitting and XPS data analysis of the core chemical shifts were used to interpret the local electronic environment of C and O atoms and to identify their bonding characteristics in fullerenols. The results of the curve fitting of C 1s and O 1s are shown in graphs B and C of FIG. 2, respectively.

To measure the chemical shifts that occur as a result of changes in the chemical bondings in fullerenols, relevant data or reference materials are generally needed. The results of the curve fitting of C 1s and O 1s of fullerenols and their assignments based on corresponding data of standards or reference materials are summarized in Table 2.

TABLE 2
Binding Energy (BE) of the fitted subpeaks and their assignment

| | Curve fitting | | Standards | Monomer (C₆₀O₅₃Mn₁₆) | |
|---|---|---|---|---|---|
| Element | BE(eV) | % | BE(eV) | Groups | Number |
| C | 289.1 | 15.12 | 287.4^{a} | O—C—O | 9 |
| (1s) | | | 289.7^{b} | | |
| | 286.5 | 16.5 | 286.7^{c} | C—O | 10 |
| | | | 287.9^{b} | | |
| | 285.2 | 69.38 | 284.5^{d} | ⅓C═C—C | 41 |
| | | | 286.1^{b} | | |
| O | 534.0 | 48.2 | 532.2^{a} | (C—O*)C═O | 9 |
| (1s) | 532.7 | 51.7 | 531.7^{e} | O—H | 10 |

^{a}1,4-benzoqionone;
^{b}fullerelon;
^{c}inositol;
^{d}C₆₀;
^{e}phenol and Standards: C. D. Wagner et al., NIST X-ray Photoelectro Spectroscopy Database, NIST Standard Reference Database 20, Version 3.3.

The C 1s region curve fitting gave three component peaks (FIG. 2, graph B). The peak (at 289.1, 15.12%) with a highest binding energy (BE) in the C 1s region is assigned to di-oxygenated carbons for two reasons: first, the carbon bonded to two oxygen atoms would have the least electron around than the carbon bonded to one oxygen atom, and as a result, to remove an electron from them would need much more BE; and second, the value of BE is much close to that of the carbon bonded to two oxygen atoms in p-benzoquinone molecules (287.4 eV). The peak with a BE at 286.5 (16.5%) is assigned to mono-oxygenated carbons, and this value is close to that of mono-oxygenated carbons in inositol (286.7 eV). The peak with a BE at 285.2 (69.38%) is assigned to unreacted carbons in the fullerenol framework. The O 1s region curve fitting gave two component peaks (FIG. 2, graph C). The peak with a higher BE at 534.0 (48.2%) is assigned to the oxygen in O—C—O groups, and the peak with a smaller BE at 523.52(51.7%), to the oxygen in hydroxyl groups.

In the reaction system only two kinds of cations can exist: one is H⁺, which is from water, and the other one is K⁺, which is from KOH. K⁺ was washed off with water after the reaction, and the XPS analysis shows that K⁺ does not exist in the composition. Fullerenol carbanions formed by OH⁻ nucleophile attack on the double bonds of fullerene are much more basic than H₂O, and they would extract H⁺ from H₂O. Accordingly, as one hydroxyl or ketone group is introduced, one hydrogen atom would be also introduced into the cage of fullerenes. From XPS studies we know that 10 hydroxyl groups and 9 ketone groups exist in the fullerenols monomer. Considering the related XPS data together with the intact framework of the fullerenols, it can be estimated that the empirical formula for the monomeric fullerenol is C₆₀H₁₉(OH)₁₀O₉ (molar mass: 1053.88 g/mol). The peaks at δ1056.20 ([C₆₀H₁₉(OH)₁₀O₉⁺.2H.]), δ2043.8874([C₆₀H₁₈O₉(OH)₁₀-C₆₀H₉O₉(OH)₇⁺]), δ2087.8369 ([C₆₀H₁₈O₉(OH)₁₀-C₆₀H₁₈O₉.(OH)₉⁺.2H.]), and δ2122.1962 ([C₆₀H₁₈O₉₋(OH)₁₀-C₆₀H₁₈O₉(OH)₁₀⁺.H₂O]) (FIG. 1, graph C) would be considered as another evidence for the monomer formula.

(4) Thermal Stability (DTA-TGA)

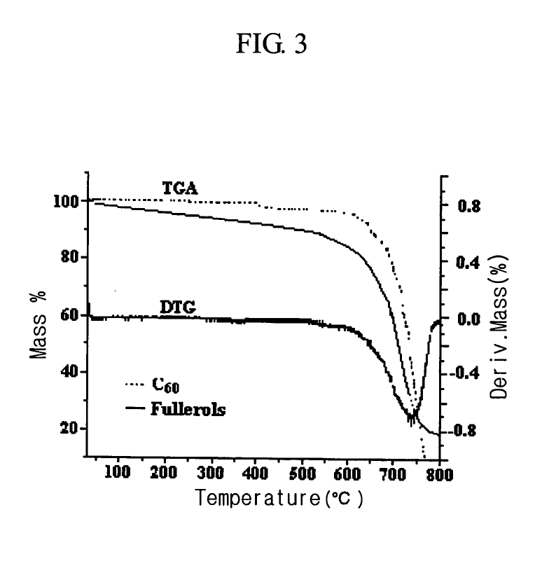

It was reported that derivatives of fullerenes having small functional groups such as —OH, —Cl, —Br, —OCH₃, and —C₆H₅ synthesized by other methods are not stable and those small groups can be easily lost from the fullerene cage under mild conditions. DTA-TGA studies were used to measure the thermal stability of the fullerenols and compared with C₆₀ (FIG. 3). As shown in FIG. 3, at below 524° C., the total mass loss of C₆₀ is 9.83%, much less than that for the fullerenols. The 6% mass loss at below 200° C. and 8% mass loss between 280 and 430° C. correspond to the elimination of physically absorbed H₂O and dehydration of polyol moieties, respectively. The fullerenols showed a mass loss of 2.97% at between 524 and 572° C., and the total mass loss is only 12.80% at below 572° C. At 766° C., the cleavage of C₆₀ is complete, and 19.97% residue of fullerenols was observed in DTG-TGA studies.

(5) HR-SEM Analysis

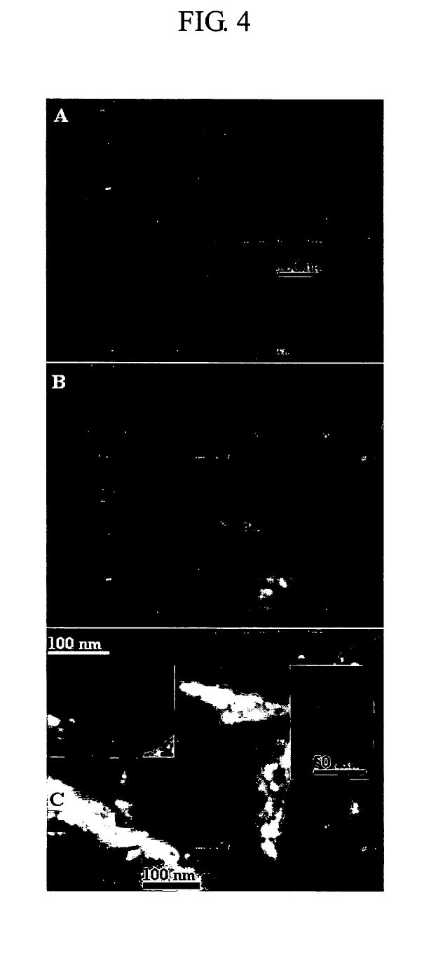

The HR-SEM images (FIG. 4) of the fullerenols products reveal that they are composed of two kinds of fullerenol particles. From FIG. 4, image A, it can be seen that fullerenol particles self-assemble together to form very ordered tiers. Image B shows unfinished tiers on the surface. From the unfinished tier, it could be clearly seen that those tiers are formed from smaller aggregates of fullerenol particles. Between those very ordered tiers, another kind of assembled fullerenol particles can be found (FIG. 4, image C). Here the aggregates look like genuine pearl chains that are composed of much smaller balls connected in tandem. Thus, the HR-SEM image studies reveal that there are two kinds of assembled figurations of fullerenol particles; one is a very ordered nanolayer and the other has a nanowire structure.

As can be seen from the above, the inventive method provides a novel, easy, one-pot, gentle, efficient and environmentally friendly method for the production of high-purity fullerenol having unique structural and physiochemical properties, which can be advantageously used in various chemical, physical and biomedical applications.

Further, the easy formation of acetyl and 2,4-dinitrophenyl-hydrazone derivatives of the inventive fullerenol shows that they can be used as a basic building block for the design of various polymers and bioactive macromolecules.

While the invention has been described with respect to the above specific embodiments, it should be recognized that various modifications and changes may be made to the invention by those skilled in the art which also fall within the scope of the invention as defined by the appended claims.

1. A method for preparing a fullerenol comprising reacting fullerene with an alkali metal hydroxide dissolved in water.

2. The method of claim 1, wherein the reaction is carried out at a temperature in the range of 50 to 150° C.

3. The method of claim 1, wherein about 0.3 to 1 mol of the alkali metal hydroxide dissolved in 5 to 100 ml of water is reacted with 1 mmol of fullerene.

4. The method of claim 1, wherein the alkali metal hydroxide is KOH or NaOH.

5. The method of claim 1, further comprising the step of adding an additional amount of water during the reaction

6. The method of claim 1, further comprising separating a solid product from the reactant, and washing and drying the product.

7. The method of claim 6, wherein the product is dried at a temperature in the range of 50 to 150° C.

8. A fullerenol having a nanolayer or nanowire structure produced by the method according to claim 1.

9. The fullerenol of claim 8, which has ten (10) hydroxyl groups and nine (9) ketone groups.
